(19) United States
(12) Patent Application Publication
(10) Pub. No.: US 2025/0297042 A1
(43) Pub. Date: Sep. 25, 2025

(54) METHOD FOR REMOVING POLYMER INSIDE OLEFIN OLIGOMERIZATION REACTOR

(71) Applicant: HANWHA TOTALENERGIES PETROCHEMICAL CO., LTD., Seosan-si, Chungcheongnam-do (KR)

(72) Inventors: Kyesung CHO, Seosan-si, Chungcheongnam-do (KR); Sang Joon OH, Seosan-si, Chungcheongnam-do (KR); Ho Sik CHANG, Yuseong-gu, Daejeon (KR); Jin Suk LEE, Dongjak-gu, Seoul (KR); Sung Mo GU, Seosan-si, Chungcheongnam-do (KR)

(21) Appl. No.: 18/862,396

(22) PCT Filed: Dec. 16, 2022
(86) PCT No.: PCT/KR2022/020562

§ 371 Date: Nov. 1, 2024

(30) Foreign Application Priority Data
Aug. 16, 2022 (KR) 10-2022-0101837

(51) Int. Cl. C08F 210/02; B01J 23/26; C08F 4/69; C08J 11/08

(52) U.S. Cl. CPC C08F 210/02; B01J 23/26; C08F 4/69; C08J 11/08

(57) ABSTRACT

Provided is a method for removing a polymer produced in an olefin oligomerization reactor. According to the method for removing a polymer of the present invention, since a polymer in a reactor may be removed within a short time, less time and cost are invested as compared with the case of disassembling the reactor and removing the polymer by manpower. In addition, downtime due to reactor maintenance decreases and productivity is improved.

### TECHNICAL FIELD

The present invention relates to a method for removing a polymer produced inside olefin oligomerization reactor.

### BACKGROUND ART

1-Hexene and 1-octene among ethylene oligomers are materials which are used in large amounts as a comonomer when polymerizing polyolefins such as polyethylene, and as production of polyolefins using a homogeneous metallocene-based catalyst increases, a demand for them is increasing steadily.

An ethylene oligomerization reaction proceeds in a stirred reactor, a tubular reactor, or a continuous stirred tank reactor, and within the reactor, a large amount of polyethylene (PE) is produced in addition to the target α-olefin, resulting in occurrence of tube blockage and fouling, which leads to inevitable process interruption, and thus, a serious problem arises in the olefin polymerization process. Specifically, polyethylene produced as a by-product forms a polymer layer and another polymer layer is formed again on the formed polymer layer, which lowers a velocity of flowing fluid, and a polymer coating layer formed along a reactor wall acts as an insulator which adversely affects heat transfer. That is, tube blockage and fouling occur, and a second treatment is needed for removing a polymer layer, which causes process shut down to occur often.

It is most effective for polymer removal when a worker enters the reactor and removes the polymer, but the method needs lots of manpower and working hours, and also requires time and economic costs for, for example, removing impurities in the reactor and replacing them with inert gas for an oligomerization reaction. Another method for removing a polymer is a removal method by peeling using a high-pressure liquid. However, a high-pressure liquid may not be directly introduced due to the structure of a device, or it is difficult to use the device when device strength is insufficient.

Thus, research on a method for removing deposited polymers by a time-economical method without opening and closing a reactor is still needed.

### DISCLOSURE

### Technical Problem

An object of the present invention is to provide a method for removing a polymer accumulated in the reactor in the latter stage of the reactor by dissolving the polymer by a time-economical method without opening and closing the reactor.

### Technical Solution

In one general aspect, a method for removing a polymer in an olefin oligomerization reactor includes: performing an olefin oligomerization reaction using a chromium-based catalyst, and removing a polymer produced in a reactor in the olefin oligomerization reaction using a solvent.

In the method for removing a polymer in an olefin oligomerization reactor according to the present invention, the solvent may be a C3 to C20 aliphatic hydrocarbon solvent or a C6 to C20 aromatic hydrocarbon solvent, and the solvent may be selected from the group consisting of toluene, xylene, chlorobenzene, dichlorobenzene, dichloromethane, hexane, methylcyclohexane, and cyclohexane.

In the method for removing a polymer in an olefin oligomerization reactor according to the present invention, the method for removing a polymer in an olefin oligomerization reactor may include: (a) performing the olefin oligomerization reaction in a first reactor using the chromium-based catalyst; (b) injecting the solvent into the first reactor which completes the olefin oligomerization reaction and heating the first reactor to produce a dissolution solution in which a polymer produced inside the first reactor is dissolved; (c) precipitating the polymer from the dissolution solution in which the polymer produced inside the first reactor is dissolved; and (d) separating and removing the precipitated polymer in a polymer removal device.

In the method for removing a polymer in an olefin oligomerization reactor according to the present invention, in (b), the solvent may be heated to 60 to 150° C., and the producing of a dissolution solution in which a polymer produced inside the first reactor is dissolved may be performed for 1 minute to 3 hours.

In the method for removing a polymer in an olefin oligomerization reactor according to the present invention, the polymer removal device may be selected from the group consisting of centrifugation, compression filtration, gravity filtration, metal filter, ceramic membrane filter, sand filter, and adsorption devices.

In the method for removing a polymer in an olefin oligomerization reactor according to the present invention, the precipitating of the polymer in (c) may be performed by a method of using a heat exchanger, a method of mixing solvents to cool the dissolution solution, or a method of treatment with a chemical, or may be performed by mixing a product discharged after performing the olefin oligomerization reaction in a reactor other than the first reactor and the dissolution solution in which a polymer produced inside the first reactor is dissolved.

In the method for removing a polymer in an olefin oligomerization reactor according to the present invention, the first reactor and the reactor other than the first reactor may be independently of each other selected from the group consisting of a batch reactor, a continuous stirred tank reactor, a tubular reactor, a loop reactor, a bubble column reactor, and a fluidized bed reactor, a temperature of the product discharged after performing the olefin oligomerization reaction in the reactor other than the first reactor may be 0 to 70° C., and a mixing volume ratio of the dissolution solution in which the polymer produced inside the first reactor is dissolved and the product discharged after performing the olefin oligomerization reaction in the reactor other than the first reactor may be 1:0.01 to 1:100.

In the method for removing a polymer in an olefin oligomerization reactor according to the present invention, after (c), separating gas and liquid in a gas-liquid separator may be further included, and after (d), separating an oligomer in a distillation device may be further included.

In another general aspect, an olefin oligomerization reaction system in which the method for removing a polymer in an olefin oligomerization reactor is performed is provided.

### Advantageous Effects

According to the method for removing a polymer of the present invention, since a polymer in a reactor may be removed within a short time, less time and cost are invested as compared with the case of disassembling the reactor and removing the polymer by manpower. In addition, downtime due to reactor maintenance decreases and productivity is improved.

### DESCRIPTION OF DRAWINGS

The above and other objects, features and advantages of the present invention will become apparent from the following description of preferred embodiments given in conjunction with the accompanying drawings, in which:

FIG. 1 is a process diagram which schematically shows a case in which there is one reactor in the olefin oligomerization reaction of the present invention.

FIG. 2 is a process diagram which schematically shows a case in which there are two or more reactors in the olefin oligomerization reaction of the present invention.

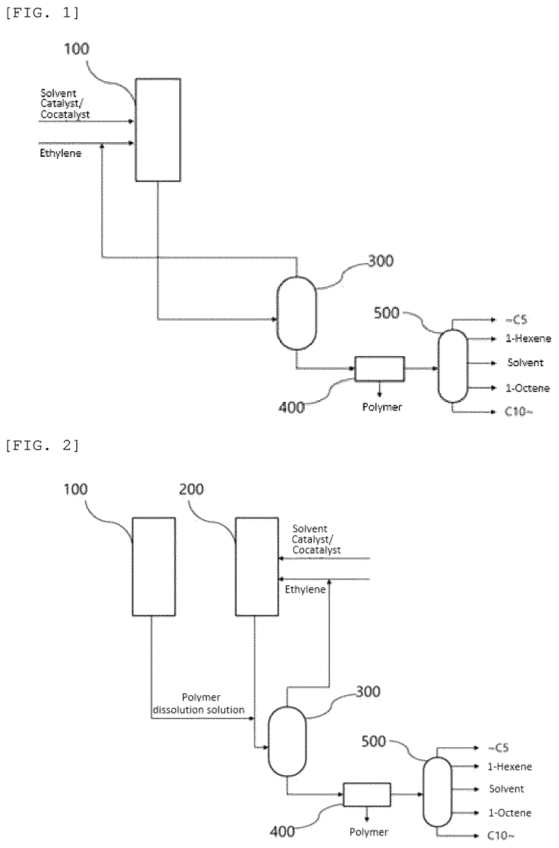

### DETAILED DESCRIPTION OF MAIN ELEMENTS

100: First reactor in which an olefin oligomerization reaction is performed
200: Second reactor in which an olefin oligomerization reaction is performed
300: Gas-liquid separator
400: Polymer removal device
500: Distillation device

### BEST MODE

Hereinafter, the method for removing a polymer in an olefin oligomerization reactor of the present invention will be described in detail with reference to the accompanying drawings.

The drawings to be provided below are provided by way of example so that the spirit of the present disclosure may be sufficiently transferred to a person skilled in the art. Therefore, the present invention is not limited to the drawings provided below but may be embodied in many different forms, and the drawings suggested below may be exaggerated in order to clarify the spirit of the present invention.

Technical terms and scientific terms used herein have the general meaning understood by those skilled in the art to which the present invention pertains, unless otherwise defined, and the description for the known function and configuration which may unnecessarily obscure the gist of the present invention will be omitted in the following description and the accompanying drawings.

In addition, the singular form used in the specification and claims appended thereto may be intended to include a plural form also, unless otherwise indicated in the context.

In the present specification and the appended claims, the terms such as “first” and “second” are not used in a limited meaning but are used for the purpose of distinguishing one constituent element from other constituent elements.

In the present specification and the appended claims, the terms such “comprise”, “include” and “have” mean that there is a characteristic or a constituent element described in the specification, and as long as it is not particularly limited, a possibility of adding one or more other characteristics or constituent elements is not excluded in advance.

In the present specification and the appended claims, when a portion such as a membrane (layer), an area, and a constituent element is present on another portion, not only a case in which the portion is in contact with and directly on another portion but also a case in which other membranes (layers), other areas, other constitutional elements, and the like are interposed between the portions is included.

The method for removing a polymer in an olefin oligomerization reactor according to the present invention is characterized by performing an olefin oligomerization reaction using a chromium-based catalyst and removing a polymer produced in the reactor in the olefin oligomerization reaction using a solvent.

In the method for removing a polymer in an olefin oligomerization reactor according to an exemplary embodiment of the present invention, the solvent used for dissolving the polymer produced in the reactor may be a hydrocarbon solvent. More specifically, the hydrocarbon solvent may be an aliphatic hydrocarbon solvent having 3 to 20 carbons (C3 to C20) or an aromatic hydrocarbon solvent having 6 to 20 carbons (C6 to C20). More specifically, the hydrocarbon solvent may be selected from the group consisting of toluene, xylene, chlorobenzene, dichlorobenzene, dichloromethane, hexane, methylcyclohexane, and cyclohexane, preferably, may be selected from the group consisting of hexane, methylcyclohexane, and cyclohexane, and more preferably, may be methylcyclohexane. When the solvent is used, polymerization activity is high, separation of the product and the solvent after the olefin oligomerization reaction is easy, and polymer dissolution is easy.

The method for removing a polymer in an olefin oligomerization reactor according to the present invention is characterized by including: (a) performing the olefin oligomerization reaction in first reactor using the chromium-based catalyst; (b) injecting the solvent into the first reactor which completes the olefin oligomerization reaction and heating the first reactor to produce a dissolution solution in which a polymer produced inside the first reactor is dissolved; (c) precipitating the polymer from the dissolution solution in which the polymer produced inside the first reactor is dissolved; and (d) separating and removing the precipitated polymer in a polymer removal device.

An olefin oligomerization system according to an exemplary embodiment of the present invention may include a first reactor 100 in which an olefin oligomerization reaction is performed, an injection line for injecting a solvent, a catalyst, and a cocatalyst, an olefin injection line, an outflow line for flowing an olefin oligomerization reaction product out of the reactor, and a polymer removal device 400 for removing a polymer in the product, and may further include a gas-liquid separator 300 for f separating an unreacted olefin and a liquid product in the product and a distillation device 500 which may separate the product.

In the method for removing a polymer in an olefin oligomerization reactor according to an exemplary embodiment of the present invention, a temperature of the solvent for dissolving the polymer produced inside the first reactor may be 60 to 150° C., preferably 70 to 140° C., more preferably 80 to 130° C., and more preferably 100 to 120° C. When the solvent is heated to the temperature in the above range and used, the polymer may be dissolved using a solvent at or higher than a melting temperature of the polymer produced in the olefin oligomerization reaction and also evaporation of the solvent is prevented.

In the method for removing a polymer in an olefin oligomerization reactor according to an exemplary embodiment of the present invention, a time for producing the dissolution solution in which the polymer produced inside the first reactor is dissolved may be 1 minute to 3 hours, preferably 3 minutes to 2 hours, more preferably 5 minutes to 1 hour, and more preferably 10 minutes to 30 minutes.

In the method for removing a polymer in an olefin oligomerization reactor according to an exemplary embodiment of the present invention, the precipitated polymer may be separated and removed in the polymer removal device, and though the type of the polymer removal device is not particularly limited, it may be selected from the group consisting of centrifugation, compression filtration, gravity filtration, metal filter, ceramic membrane filter, sand filter, and adsorption devices and used.

In the method for removing a polymer in an olefin oligomerization reactor according to an exemplary embodiment of the present invention, the precipitation of the polymer in (c) may be performed by a method of using a heat exchanger, a method of mixing solvents and cooling the dissolution solution, or a method of treatment with a chemical, in which the chemical includes alcohol or water. More specifically, the precipitation of the polymer in (c) may be performed by mixing the product discharged after performing the olefin oligomerization reaction in the reactor other than the first reactor and the dissolution solution in which the polymer produced inside the first reactor is dissolved. That is, while the dissolution solution including the polymer produced inside the first reactor is produced by injecting a solvent into the first reactor in which the olefin oligomerization reaction is completed and heating, the olefin oligomerization reaction may proceed in the reactor other than the first reactor, and the product discharged after performing the olefin oligomerization reaction in the reactor other than the first reactor and the dissolution solution in which the polymer produced inside the first reactor is dissolved may be mixed to precipitate the polymer.

Therefore, the olefin oligomerization system according to an exemplary embodiment of the present invention may include a first reactor 100 in which an olefin oligomerization reaction is performed, a second reactor 200 in which an olefin oligomerization reaction is performed, an injection line for injecting a solvent, a catalyst, and a cocatalyst, an olefin injection line, an outflow line for flowing an olefin oligomerization reaction product out of the reactor, and a polymer removal device 400 for removing a polymer in the product, and may further include a gas-liquid separator 300 for separating an unreacted olefin and a liquid product in the product and a distillation device 500 which may separate the product.

In the method for removing a polymer in an olefin oligomerization reactor according to an exemplary embodiment of the present invention, the first reactor in which the olefin oligomerization reaction is performed and the reactor other than the first reactor may be independently of each other a reactor selected from the group consisting of a batch reactor, a continuous stirred tank reactor, a tubular reactor, a loop reactor, a bubble column reactor, and a fluidized bed reactor, and preferably, may be a continuous stirred tank reactor or a tubular reactor, and more preferably, may be a continuous stirred tank reactor.

In the method for removing a polymer in an olefin oligomerization reactor according to an exemplary embodiment of the present invention, a temperature of the product discharged after performing the olefin oligomerization reaction in the reactor other than the first reactor may be 0 to 70° C., preferably 5 to 50° C., and more preferably 10 to 45° C. In addition, a mixing volume ratio of the dissolution solution including the polymer produced inside the first reactor and the product discharged after performing the olefin oligomerization reaction in the reactor other than the first reactor may be 1:0.01 to 1:100, preferably 1:0.5 to 1:50, and more preferably 1:5 to 1:20. When the temperature and the volume ratio are satisfied, polymer precipitation is easily performed.

In the method for removing a polymer in an olefin oligomerization reactor according to an exemplary embodiment of the present invention, after precipitating the polymer, separating gas and liquid in a gas-liquid separator may be further included, and after separating and removing the precipitated polymer in the polymer removal device, separating an oligomer in a distillation device may be further included. That is, the product produced by the olefin oligomerization reaction in the first reactor may pass through the gas-liquid separator to remove a small amount of polymer in the polymer removal device and be separated into each product in the distillation device.

Therefore, the olefin oligomerization reaction system according to the present invention is characterized by performing the method for removing a polymer in the olefin oligomerization reactor.

Hereinafter, the present invention will be described in detail by the examples. However, the examples are for describing the present invention in more detail, and the scope of the present invention is not limited to the following examples.

### Example 1

Methylcyclohexane at a rate of 1.5 L/hr, ethylene at a rate of 250 g/hr, a catalyst at a rate of 0.02 μmol/hr, and a cocatalyst at a rate of 7 μmol/hr were injected into a 2 L CSTR reactor heated to 40° C. The reaction was performed for 90 hours in a state of maintaining a reaction temperature at 40° C. while continuously supplying ethylene so that reactor internal pressure was maintained at 30 bar, and then reactant injection was blocked, the product inside the reactor was discharged, and the reaction was completed.

After the reaction, in order to remove the polymer produced inside the reactor, methylcyclohexane was injected into the reactor, the temperature was raised to 110° C., and the polymer was dissolved for 20 minutes. Thereafter, a polymer dissolution solution at a high temperature was discharged from the reactor and mixed with an oligomerization product (40° C.) produced in the other reactor at a volume ratio of 1:10 to precipitate the polymer in a fluff form. Thereafter, the precipitated polymer was removed using a centrifuge.

### Comparative Example 1

A polymer was precipitated in the same manner as in Example 1, except that the polymer was dissolved for 60 minutes by raising the temperature to 90° C., instead of dissolving the polymer for 20 minutes by raising the temperature to 110° C.

### Comparative Example 2

A polymer was precipitated in the same manner as in Example 1, except that decaline was used as a solvent for dissolving the polymer, instead of methylcyclohexane.

### Comparative Example 3

A polymer was precipitated in the same manner as in Example 1, except that decaline was used as a solvent for dissolving the polymer, instead of methylcyclohexane, and the polymer was dissolved by raising the temperature to 130° C., instead of dissolving the polymer by raising the temperature to 110° C.

### Comparative Example 4

A polymer was precipitated in the same manner as in Example 1, except that xylene was used as a solvent for dissolving the polymer, instead of methylcyclohexane.

### Comparative Example 5

A polymer was precipitated in the same manner as in Example 1, except that xylene was used as a solvent for dissolving the polymer, instead of methylcyclohexane, and the polymer was dissolved by raising the temperature to 130° C., instead of dissolving the polymer by raising the temperature to 110° C.

### Comparative Example 6

A polymer was precipitated in the same manner as in Example 1, except that the polymer was precipitated by mixing the polymer dissolution solution and the oligomerization product (40° C.) produced in the other reactor at a volume ratio of 10:1, instead of 1:10.

### Comparative Example 7

A polymer was precipitated in the same manner as in Example 1, except that the polymer was precipitated by mixing the polymer dissolution solution and the oligomerization product (40° C.) produced in the other reactor at a volume ratio of 1:1, instead of 1:10.

### <Experimental Example> Evaluation of Amount of Precipitated Polymer

Amounts of the polymers precipitated according to the polymer precipitation methods of Example 1 and Comparative Examples 1 to 7 are shown in the following Table 1:

TABLE 1

| | | | | Whether | Mixing | |
|---|---|---|---|---|---|---|
| | | | | polymer | ratio | Amount (g) of |
| | | Temperature | Time | was | (volume | precipitated |
| | Solvent | (° C.) | (min) | dissolved | ratio) | polymer |
| Example 1 | Methylcyclo- | 110 | 20 | ∘ | 1:10 | 10.2 |
| | hexane | | | | | |
| Comparative | Methylcyclo- | 90 | 60 | x | | |
| Example 1 | hexane | | | | | |
| Comparative | Decaline | 110 | 20 | x | | |
| Example 2 | | | | | | |
| Comparative | Decaline | 130 | 20 | ∘ | 1:10 | 9.5 |
| Example 3 | | | | | | |
| Comparative | Xylene | 110 | 20 | x | | |
| Example 4 | | | | | | |
| Comparative | Xylene | 130 | 20 | ∘ | 1:10 | 9.7 |
| Example 5 | | | | | | |
| Comparative | Methylcyclo- | 110 | 20 | ∘ | 10:1 | 3.2 |
| Example 6 | hexane | | | | | |
| Comparative | Methylcyclo- | 110 | 20 | ∘ | 1:1 | 7.8 |
| Example 7 | hexane | | | | | |

As shown in Table 1, when decaline or xylene was used as the solvent, other than methylcyclohexane, the polymer was not dissolved at 110° C. When the solvent was used, a high temperature of 130° C. was needed for dissolving the polymer.

In addition, when the polymer was precipitated at a mixing volume ratio of 10:1, the polymer dissolution solution was not sufficiently cooled, so that a polymer precipitation amount was very small. In this case, the dissolution solution transferring to a distillation column in the latter stage is in a state in which the polymer is not completely removed in the centrifuge, resulting in fouling.

In conclusion, according to the method for removing a polymer of the present invention, since a polymer in a reactor may be removed within a short time, less time and cost are invested as compared with the case of disassembling the reactor and removing the polymer by manpower. In addition, downtime due to reactor maintenance decreases and productivity is improved.

1. A method for removing a polymer in an olefin oligomerization reactor, the method comprising: performing an olefin oligomerization reaction using a chromium-based catalyst, and removing a polymer produced in a reactor in the olefin oligomerization reaction using a solvent.

2. The method for removing a polymer in an olefin oligomerization reactor of claim 1, wherein the solvent is a C3 to C20 aliphatic hydrocarbon solvent or a C6 to C20 aromatic hydrocarbon solvent.

3. The method for removing a polymer in an olefin oligomerization reactor of claim 2, wherein the solvent is selected from the group consisting of toluene, xylene, chlorobenzene, dichlorobenzene, dichloromethane, hexane, methylcyclohexane, and cyclohexane.

4. The method for removing a polymer in an olefin oligomerization reactor of claim 1, wherein the method for removing a polymer in an olefin oligomerization reactor includes:
(a) performing the olefin oligomerization reaction in a first reactor using the chromium-based catalyst;
(b) injecting the solvent into the first reactor which completes the olefin oligomerization reaction and heating the first reactor to produce a dissolution solution in which a polymer produced inside the first reactor is dissolved;
(c) precipitating the polymer from the dissolution solution in which the polymer produced inside the first reactor is dissolved; and
(d) separating and removing the precipitated polymer in a polymer removal device.

5. The method for removing a polymer in an olefin oligomerization reactor of claim 4, wherein in (b), the solvent is heated to 60 to 150° C.

6. The method for removing a polymer in an olefin oligomerization reactor of claim 4, wherein in (b), the producing of a dissolution solution in which a polymer produced inside the first reactor is dissolved is performed for 1 minute to 3 hours.

7. The method for removing a polymer in an olefin oligomerization reactor of claim 4, wherein the polymer removal device is selected from the group consisting of centrifugation, compression filtration, gravity filtration, metal filter, ceramic membrane filter, sand filter, and adsorption devices.

8. The method for removing a polymer in an olefin oligomerization reactor of claim 4, wherein the precipitating of the polymer in (c) is performed by a method of using a heat exchanger, a method of mixing solvents to cool the dissolution solution, or a method of treatment with a chemical.

9. The method for removing a polymer in an olefin oligomerization reactor of claim 4, wherein the precipitating of the polymer in (c) is performed by mixing the product discharged after performing the olefin oligomerization reaction in the reactor other than the first reactor and the dissolution solution in which the polymer produced inside the first reactor is dissolved.

10. The method for removing a polymer in an olefin oligomerization reactor of claim 9, wherein the first reactor and the reactor other than the first reactor are independently of each other selected from the group consisting of a batch reactor, a continuous stirred tank reactor, a tubular reactor, a loop reactor, a bubble column reactor, and a fluidized bed reactor.

11. The method for removing a polymer in an olefin oligomerization reactor of claim 9, wherein a temperature of the product discharged after performing the olefin oligomerization reaction in the reactor other than the first reactor is 0 to 70° C.

12. The method for removing a polymer in an olefin oligomerization reactor of claim 9, wherein a mixing volume ratio of the dissolution solution in which the polymer produced inside the first reactor is dissolved and the product discharged after performing the olefin oligomerization reaction in the reactor other than the first reactor is 1:0.01 to 1:100.

13. The method for removing a polymer in an olefin oligomerization reactor of claim 4, further comprising: after (c), separating gas and liquid in a gas-liquid separator.

14. The method for removing a polymer in an olefin oligomerization reactor of claim 4, further comprising: after (d), separating the oligomer in a distillation device.

15. An olefin oligomerization reaction system in which the method for removing a polymer in an olefin oligomerization reactor of claim 1 is performed.
